(19) United States
(12) Patent Application Publication
(10) Pub. No.: US 2025/0000506 A1
(43) Pub. Date: Jan. 2, 2025

(54) IMPLANTABLE FLEXIBLE TISSUE ATTACHMENT DEVICE

(71) Applicant: BIOVENTUS, LLC, Durham, NC (US)

(72) Inventors: Mitchell VANDERLAAN, Durham, NC (US); Ryan KOCH, Durham, NC (US); Lawrence BOYD, Durham, NC (US); James Christopher TEAGUE, Durham, NC (US)

(21) Appl. No.: 18/756,114

(22) Filed: Jun. 27, 2024

Related U.S. Application Data
(60) Provisional application No. 63/524,283, filed on Jun. 30, 2023.

(51) Int. Cl. A61B 17/04; A61F 2/08; A61L 31/04

(52) U.S. Cl. CPC A61B 17/0466; A61B 17/0401; A61L 31/044; A61B 2017/0453; A61B 2017/0464; A61F 2/0811

(57) ABSTRACT

An implantable device to fix tissue to a bone to assist with tissue regrowth after fixation is provided. The device is an elongate flexible member, wherein the flexible member comprises a plurality of layers that extend along a length of the device. The flexible member includes a central portion, first and second intermediate portions outboard of respective opposite first and second ends of the central portion, and a first end portion that is outboard of the first intermediate portion and a second end portion that is outboard of the second intermediate portion. The elongate flexible member comprises two outer layers. A woven layer is disposed between the two elongate outer layers. The first and second intermediate portions establish a first width along a majority of their length, and wherein the first width is greater than a width of the central portion and a width of the entire woven layer.

### CROSS-REFERENCE TO RELATED APPLICATIONS

This application claims priority from U.S. Provisional application No. 63/524,283, filed Jun. 30, 2023, the entirety of which is hereby incorporated by reference herein.

### TECHNICAL FIELD

The disclosure relates medical devices and techniques directed to repair damaged soft tissue. Conventionally, techniques have been used to repair or reconnect soft tissue by suturing the soft tissue to a fixed point (such as a bone), typically with bone anchors. Once the soft tissue is mechanically fixed in the correct position, healing occurs with tissue regrowth in the correct position. Typically, mechanical repair solutions that use only sutures have been noted to have outcomes that risk future damage in the same way or similar ways. This disclosure is directed to a device that can be implanted to result in permanent repair of soft tissue, which facilitates soft tissue regrowth in correct orientation.

### SUMMARY

This disclosure relates to a device that enhances repair or restoration of soft tissue damage and specifically allows for mechanical fixation of soft tissue and promotes regrowth of the soft tissue after placement.

A first representative embodiment is provided. The embodiment includes an implantable tissue attachment device. The device includes an elongate flexible member, wherein the flexible member comprises a plurality of layers that extend along a length of the device, wherein the flexible member includes a central portion, first and second intermediate portions extending from respective opposite first and second ends of the central portion, and a first end portion that extends from the first intermediate portion and a second end portion that is extends from the second intermediate portion. The elongate flexible member comprises two elongate layers that are formed at least in part from a collagen material, wherein the two elongate layers are disposed to establish opposite outer layers of the flexible member. A woven layer is disposed between the two elongate collagen layers, wherein the first and second intermediate portions establish a first width along a majority of their length, and wherein the first width is greater than a width of the central portion along at least a majority of the length of the first and second intermediate portions. The first end portion transitions from the first intermediate portion and the width of the first end portion narrows along a portion the first end portion until reaching a minimum width at an outer tip of the first end portion. The second end portion transition from the second intermediate portion and the width of the second end portion narrows along a portion of the second end portion until reaching a minimum width at an outer tip of the second end portion.

Another representative embodiment of the disclosure is provided. The embodiment includes a method of repairing a tissue to bone connecting comprises providing an implantable tissue attachment device of the embodiment of the paragraph directly above, or any of Numbered Paragraphs 1-36, below. The method further includes the following steps:

preparing a center bone aperture and first and second bone apertures that are each offset laterally and proximal to the central bone aperture on opposite sides of the center bone aperture;
threading a first end portion of the flexible member through an eyelet of a first anchor until central portion of the flexible member is centered through the eyelet;
inserting and fixing the first anchor in the center bone aperture;
pulling the first end portion through a first position of the tissue and pulling the second end portion through a second position of the tissue at a laterally offset position from the first position;
pulling the first and second end portions toward the respective first and second offset lateral bone apertures, arranging the flexible member such that the respective first and second intermediate portions lie substantially flat upon spaced apart respective outwardly facing surfaces of the tissue with surface to surface contact therebetween;
threading the first end portion through an eyelet of a second anchor and inserting the second bone anchor into the first offset lateral bone aperture;
threading the second end portion through an eyelet of a third anchor and inserting the third bone anchor into the second offset later bone aperture;
simultaneously with threading the first end portion through the eyelet of the second anchor, pulling the first end portion that extends through the eyelet of the second anchor to create tension in the first intermediate portion;
simultaneously with threading the second end portion through the eyelet of the third anchor, pulling the second end portion that extends through the eyelet of the third anchor to create tension in the second intermediate portion;
fixing the second anchor into the first offset lateral bone aperture and arranging the first end portion such that at least one of the outer surfaces of the first end portion makes contact with bone within the first offset lateral bone aperture; and fixing the third anchor into the second offset lateral bone aperture and arranging the second end portion such that at least one of the outer surfaces of the second end portion makes contact with bone within the second offset lateral bone aperture.

According to one embodiment, disclosed is a composite implantable tissue attachment device that can be attached to tissue during a surgical procedure. An attachment device as disclosed herein can include a mechanical reinforcing component and a cellular scaffold component affixed thereto. A tissue attachment device as disclosed herein can define a length, width and depth. In addition, at least a portion of the length of the device can define a width that is at least about 1 millimeter across, and this width can be greater than the depth of the device along this length. A device can also include narrower sections, for instance narrower ends for instance to aid in delivery of a device to a repair site. This reduced width allows for position adjustment to suit variations in patient anatomy while maintaining the key features of the mechanical reinforcement and tissue regrowth and securing fixation where required for tissue attachment.

### BRIEF DESCRIPTION OF THE DRAWINGS

FIG. 1 is a perspective view of a flexible tissue attachment device configured to be used in a procedure to reattach a flexible member (such rotator cuff) to a bone.

FIG. 1a is a perspective view of another flexible tissue attachment device, depicting schematically the running and the lateral stitches used to assemble the device as well as lateral stitches to provide position markings.

FIG. 2 is a top view of the device of FIG. 1, the bottom view (which would depict layer 40 instead of layer 20) is the same. The view includes an alternate possible transition (320c) between the intermediate portion and the end portion than was depicted in FIG. 1.

FIG. 3 is a top view of the first outer layer of the device of FIG. 1. The second outer layer is the same.

FIG. 4 is a top view of an internal layer of the device. The layer is depicted at a specific length but the layer may be indeterminate length such that it can extend further outside of the outer layers (20, 40) than depicted in FIGS. 1, 1a, and 2.

FIG. 4a is a top view of an alternate internal layer of the device. The layer is depicted at a specific length but the layer may be of indeterminate length such that it can extend further outside of the layers (20, 40) than depicted in FIGS. 1, 1a, and 2.

FIG. 4b is a top view of a device that is constructed with the layer of FIG. 4a, with the geometry of the layer depicted in broken lines for comparison between the geometry of the outer layers and the layer of FIG. 4a.

FIG. 5 is a detail view of detail B of FIG. 2.

FIG. 6 is a detail view of detail A of 3.

FIG. 7a is a detail view of the end portion of the device with the inner layer (80) removed, depicting the stitching lines approaching and leaving the tip of the end portion at an acute angle with respect to each other.

FIG. 7b a detail view of the end portion of the device with the inner layer (80) removed, depicting the stitching lines approaching and leaving the tip of the end portion in parallel to each other.

FIG. 7c is a detail view of the end portion of the device with the inner layer (80) removed, depicting the stitching lines making a perpendicular angle with a section of vertical stitching (242) most proximate to the tip of the end portion.

FIG. 8a is a detail view of the end portion of the outer layer depicting the edges of the layer approaching the tip with straight lines that are at an acute angle with respect to each other.

FIG. 8b is a detail view of the end portion of the outer layer depicting the edges of the layer leaving the tip with curved geometry and then transition to straight lines further away from the tip, with the straight lines at an acute angle with respect to each other.

FIG. 9 is schematic view of the device of FIG. 1 or 1a being deployed clinically, with the central portion of the device extended within an eyelet of a bone anchor with the central portion centered about the eyelet such that the lateral stitches are outboard of both sides of the eyelet.

FIG. 9a is a top view of a portion of the image of FIG. 9 depicting the lateral stitches outboard of both sides of the eyelet.

FIG. 10 is a schematic view of the device with the center bone anchor fixed to the bone (B) and the portions of the device extending through a tissue (C) desired to be fixed with respect to the bone.

FIG. 11 is a schematic view of the device with the first and second end portions of the device extending through eyelets of spaced bone anchors offset from an end of the tissue (C), with the two bone anchors fixed to the bone, and depicting sutures (700, 710, 720, 730) also passing through the tissue and eyelets of the bone anchors for additional support of the tissue in parallel to the device.

FIG. 11a is a schematic view of a deployed device, depicting the end portions (380, 400) of the device extending into the bone holes (4000) (Z, Y) to enhance contact between the first and second layers (20, 40) of the device and the bone.

FIG. 12 is a top view of the device implanted within a patient in a rotator cuff procedure with the device deployed along with sutures in a double row fixation technique.

FIG. 13 is a view of a step of implanting the an end portion of the device into the eyelet of an anchor as well as two sutures in order to fix one of the ends of the device into the tissue during the double row fixation technique of FIG. 12.

FIG. 13a is a detail view showing a deployed central portion of the device extending through the eyelet of an anchor as well as well as two sutures in order to deploy the eyelet and the anchor into a bone hole (4003) during performance of the double row fixation technique of FIG. 12.

FIG. 14 is view of a deployed end portion of the device extending through an eyelet of an anchor, along with two sutures, and positioned to be inserted into a bone hole for a double row fixation technique.

FIG. 15 is a side cross-sectional view of the end portion of the device as well as two sutures extending through an eyelet of an anchor that is deployed within a bone aperture, with the intermediate portion of the device pressing the muscle into the bone as depicted schematically with a force from the device upon the muscle (F4) and a force of the muscle upon the bone (F5), with the central portion of the device retained by a second anchor within the bone to form a portion of the double row fixation technique of FIG. 12.

### DETAILED DESCRIPTION OF THE DRAWINGS

Presently disclosed subject matter is generally directed to implantable devices as may be beneficially utilized in tissue repair protocols such as, without limitation, tissue replacement, stabilization, reconstruction, and the like. More specifically, disclosed devices can be affixed to one or more tissues. For example, an implantable device as disclosed herein can be attached to one or more tissues through a stitching mechanism. Also disclosed herein are methods for forming the devices as well as methods for using the devices.

At least a portion of a disclosed device can be relatively wide, e.g., wider than materials that have been commonly utilized for soft tissue repair in the past. As a result, disclosed devices can cover a larger surface area of a tissue to which they are applied. This greater area of contact can distribute the load of the repair materials over a larger area of the tissue to which it is affixed and prevent pullout and repetition of or additional damage to a site. In addition, the greater area of contact between a device and tissue to which it is applied can increase area of contact between tissues that are approximated during the procedure, e.g., can better restore the natural tendon to bone footprint in a tendon repair procedure.

Further, portions of the end portions 180, 200; 380, 400 (discussed herein) may be sized to extend into bone holes (4000) along with the bone anchors (800) such that the end portions 180, 200; 380, 400 of the device 10, and specifically the first and second layers 20, 40 of the device make contact with the bone to allow for enhanced connection and tissue regrowth from the bone (B) to the tissue (C). Accordingly, utilization of disclosed devices can improve biological interaction between approximated tissues and encourage healing of the tissues, for instance through enhanced contact area with the vascular supply due to both enhanced contact area and improved load distribution. In addition, devices as disclosed herein can provide improvements in maneuverability, strength, tenacity, and/or immediate reinforcement ability of suture-type materials. Disclosed devices also combine these capabilities with the tissue regeneration and excellent long-term healing characteristics of cellular scaffold materials.

Implantable devices as disclosed herein can, in general, be utilized in any fashion as is known for suture materials. In contrast to suture, however, disclosed devices define a shape more conducive to a wide variety of repair and reconstructive procedures. In particular, disclosed devices can have a width that can improve contact between the device and tissue to which it is applied. In addition, a cellular scaffold component of disclosed devices can be less abrasive on surrounding tissue than suture, reducing the likelihood that a device will pull out of the tissue. Implantable devices as disclosed herein can be utilized in any fashion as is known for surgical tapes, surgical meshes, and the like, including tissue fixation devices. This implantable devices can be easily incorporated into the current standard practice for placement of bone anchors, with the added benefit that the design allow for length adjustment by allowing translation in the eyelet not possible with the prior art designs with increased width distal ends.

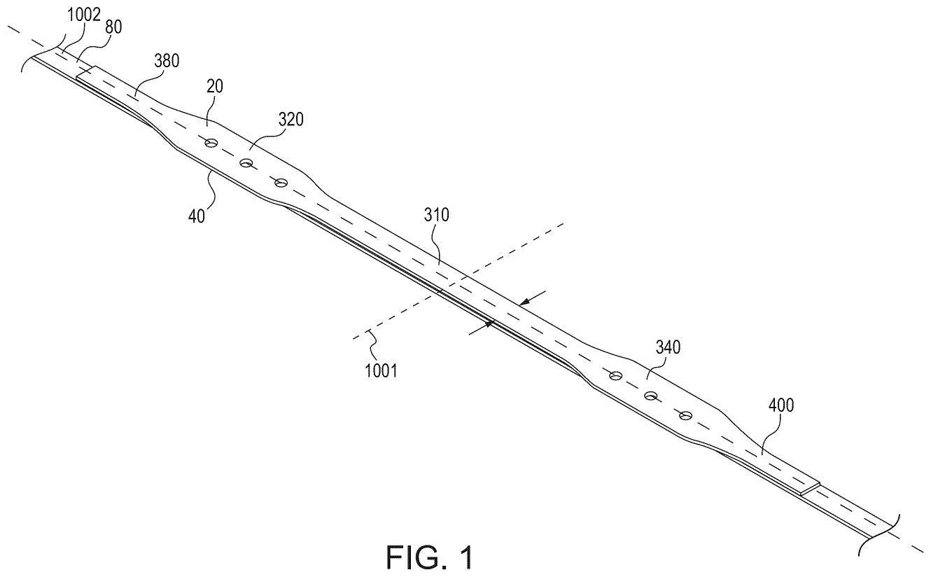

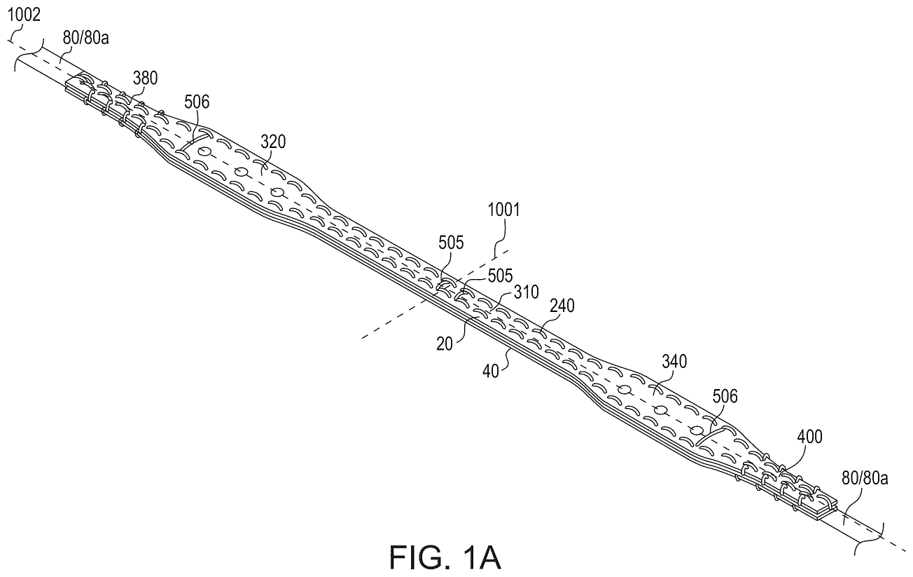

Turning now to FIGS. 1-8, an implantable attachment device 10 is provided. The device 10 is configured to be used in one or more anatomical clinical procedures, such as a rotator cuff repair. One of ordinary skill in the art will appreciate the attachment device 10 is configured to be used other clinical procedures where one or more anatomical features are desired to be connected to or with respect to other anatomical features such that one or more degrees of freedom of relative movement is provided between the anatomical features. For the sake of brevity, this specification is drafted to specifically discuss clinical procedures using the attachment device for a rotator cuff repair procedure, such as clinical procedures identified as partial rotator cuff repair, full rotator cuff repair, and massive rotator cuff repair. The current device is focused on enabling the use of suture anchors, which are commonly used to secure soft tissue to underlying bone. Often these are tendons connecting between muscles and their bony insertion/anchor points. The rotator cuff is a key tendon that can be surgically repaired. Another commonly repaired tendon includes the Achilles tendon connecting the calf muscles (gastrocnemius and soleus) to the heel (calcaneus) bone at the back of the foot. Other applications are found in the hand, elbow, wrist and lower limb joints such as the knee as well as ankle later stabilization. Ligaments may also be reapproximated to bone using a device such as that disclosed. The disclosed device (with clinically appropriate geometry changes such as length or width changes of the various portions) can be successfully used for ACL, PCT, MCL, or LCL repair. One of ordinary skill in the art after a thorough review and understanding of this specification will readily understand how the attachment device may be implemented for other clinical procedures. Any modifications of the attachment device 10 that are needed to adapt the device for another clinical procedure will be readily understood and would be possible to be performed by one of ordinary skill in the art after a thorough review and understanding of this specification with only routine optimization and without undue experimentation.

In a representative embodiment, the device 10 is configured to be used with a lateral double row fixation technique. In this technique, the intermediate portions 120, 140 (discussed below) cross each other when implanted. In another representative embodiment, the device 10 is configured to be used in a medial fixation technique, where a central portion 110 (discussed below) extends through an anchor that is between two anchors fixed to the humerus laterally in the lateral double row fixation technique, and the intermediate portions extend to the respective anchors in the humerus distally such that the two intermediate portions 120, 140 do not cross each other. In embodiments where the device 10 is used in either a medial fixation technique or a double row lateral fixation technique, sutures may also be used to attach the tendon to the humerus in a conventional double row lateral fixation technique.

The attachment device 10 includes a plurality of layers and a changing width along its length. The term length is the direction of the device in the direction that a device would be pulled through an eyelet through a typical bone anchor that is used to fix sutures and the device in a fixation technique (such as double row fixation or medial fixation techniques. The term width is a direction perpendicular to the length and extends along the same plane as the length direction. The term depth is the thickness of the device (or a specific layer of the device) and the depth is perpendicular to the length and the width. The depth extends along a plane that is perpendicular to the plane that includes both the length and width directions.

In some embodiments, and in an exemplary embodiment discussed herein and depicted in the figures, the device 10 has three layers that are disposed together in face to face contact such the plane that includes the length and width directions of each layer are parallel to each other (as the device 10 lays flat). In other embodiments, the device 10 may have additional layers, such as 5 layers (with three alternating collagen layers and two woven layers, as discussed in detail below, disposed between the alternating collagen layers). In other embodiments, the device 10 may include two woven layers that are provided next to each other with collagen layers provided outboard of the two woven layers. In some embodiments, one or more of the three layers may include several layers of the same material that are fixed together (bonded, stitched, or pressed together) to establish a single functional layer. The device is configured with two reabsorbable layers that establish a suitable scaffold for tissue regrowth in order to assist with strengthening the tissue that is being repaired.

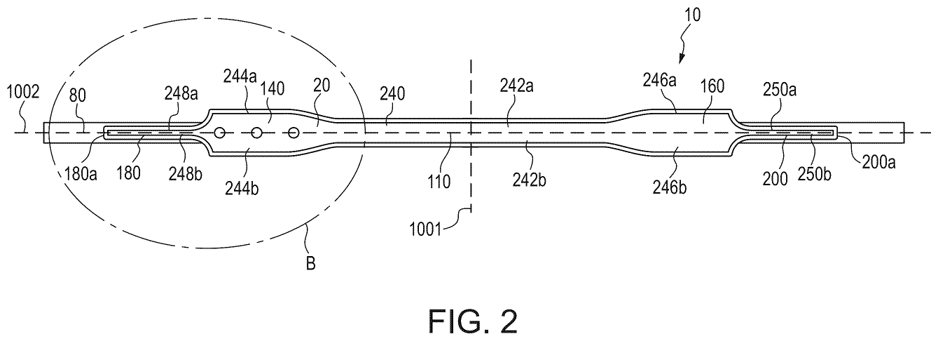

Turning now to FIGS. 1 and 2, the device 10 may include first and second outer layers 20, 40 and an intermediate layer 80 that extends between the first and second outer layers 20, 40. The three layers 20, 80, 40 (in the order that they are arranged to form the device 10) may be fixed together to form a unitary member 10 such that all three layers move and bend together.

The device 10 may include a central portion 110, first and second intermediate portions 140, 160 that are disposed outboard of the opposite ends of the central portion 110, and first and second end portions 180, 200 that extend outboard of the opposite end of each respective intermediate portion 140, 160 from the end that extends from the central portion 110. The geometry of the device 10 changes between the central 120, intermediate 140, 160, and end portions 180, 200.

In some embodiments, the first and second outer layers 20, 40 are formed from the same material. The first and second outer layers 20, 40 may be a uniform material along their length. The intermediate layer 80 is a different material than the first and second layers 20, 40.

In a preferred embodiment, the three layers are stitched together with a line 240 of stitching that circumnavigates the entire or at least a majority of the perimeter of the device 10. The line 240 of stitching may be formed with a running stitch, or any other type of stitching that is known in the art to fix several biomedical flexible materials together. In some embodiments, the layers are mattress stitched together. In preferred embodiments, the line 240 of stitching will be close to the perimeter of the entire device 10 and specifically will extend at a constant or a substantially constant distance from the outer edge of the device 10. The constant or substantially constant distance may extend around the entire perimeter of the device, or in other embodiments, there may be different distances around the perimeter of the device that is at different features of the device, as discussed below. The term “substantially constant” is defined herein to include a desired nominal distance as well as plus or minus 20% tolerance on the distance. In some embodiments, the constant distance is 1.0 mm, with a tolerance of +1.0 mm, and −0 mm.

The line 240 of stitching can be with conventional sutures or other biocompatible materials that are thin, flexible and strong in tension. Any suture material as is known in the art can be utilized. Suture material for an implantable device can be absorbable or non-absorbable, as desired. Suture can be of any size (e.g., from #11-0 up to #5 in size), suture can be multifilament and braided or twisted, or can be mono-filament. Suture can be sterile or non-sterile, of natural, synthetic, or a combination of materials. In one embodiment, suture material can be coated. Typical coatings can include, for example, collagen, magnesium stearate, PTFE, silicone, polybutilate, and antimicrobial substances.

A large variety of suitable suture is known to those of skill in the art and can include, without limitation, collagen, catgut, polyglycolic acid, polyglactin 910, poliglecaprone 25, polydioxanone, surgical silk, surgical cotton, nylon, polybutester, polyester fibers, polyethylene fibers, polypropylene fibers, Polydek-such as 5-0 Polydek and the like. Other sizes than 5-0 can be used, such as 4-0 or 3-0, or 2-0. For instance, polyethylene suture such as co-braided polyethylene suture can be utilized.

Because the line 240 of stitching extends about the perimeter of the device 10 there are two lines of stitching that extend through each portion of the device. The term extends from here is means that the line 240 moves between various portions within the device, and covers the line 240 between the various components regardless of the direction that the line 240 is stitched onto the device, and regardless of a location where the first stitch was provided and a location where the final stitch was provided. Similarly the term returns to means that the line ultimately reaches the location mentioned regardless of whether the first or last stitch is at that location, or whether the location is between the location of the first or last stitches.

The central portion has first and second lines 242a, 242b that extend therethrough, the first intermediate portion 140 has first and second lines 244a, 244b that extend therethrough, the second intermediate portion 160 has first and second lines 246a, 246b that extend therethrough. In some embodiments, the first and second end portions 180 and 200 each have first and second lines 248a, 248b, 250a, 250b that extend therethrough. In some of these embodiments, the first and second lines 248a, 248b may intersect (such that there is no space between the portions of the first and second lines 248a, 248b that intersect) and the first and second lines 250a, 250b within the second end portion 200 may also intersect (such that there is no space between portions of the first and second lines 250a, 250b that intersect).

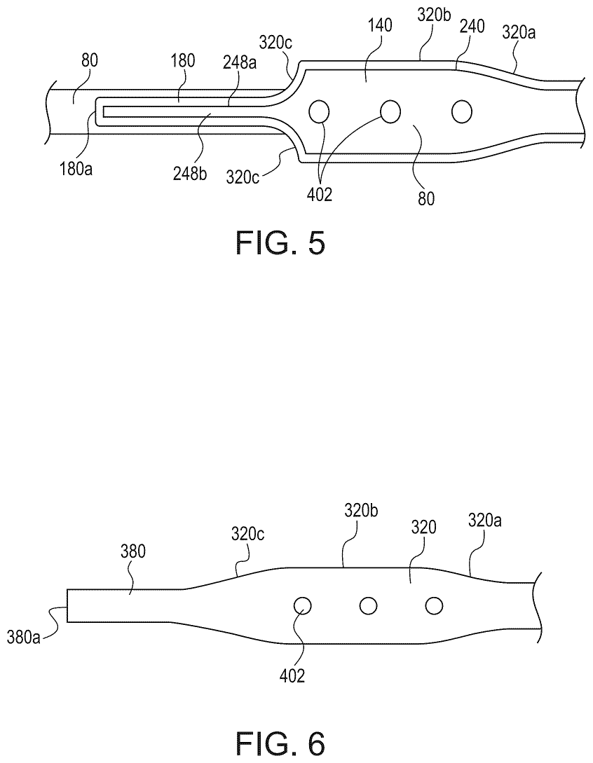

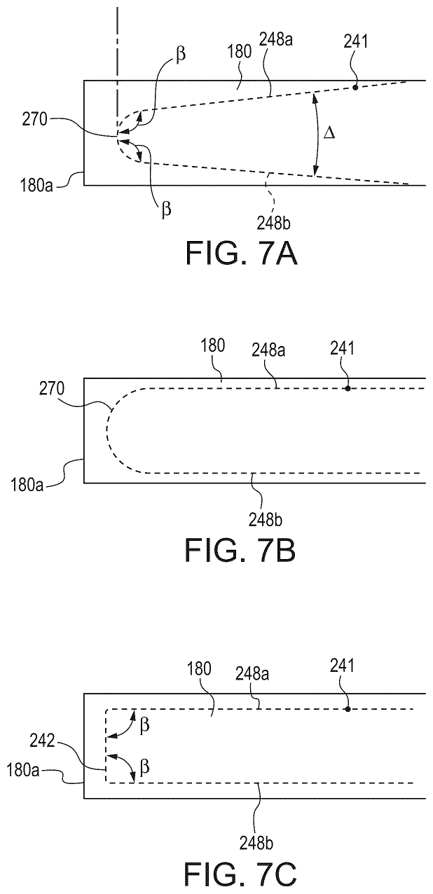

In some embodiments, the first and second lines 248a, 248b, 250a, 250b within the respective first and second end portions 180, 200 intersect proximate to the end tip 180a, 200a of the end portion (FIGS. 5, 7a-7c). The intersection may via a curve 270 that transitions between the two lines within the end portion, or it may there may be a portion that is straight but at an angle β to the approaching first and second line. The angle may be 90 degrees (potentially with a minor filet as needed to allow for a continuous stitch needed to change directions by ninety degrees), or the angle β may be an obtuse (measured with respect to a direction extending toward the neighboring intermediate portion 140/160). In this possibility, the two lines 248a/248b approach each other at an acute angle Δ with respect to each other.

As will be discussed further herein, the lines 244a, 244b, 246a, 246b that extend through the respective first and second intermediate portions 140, 160 may be, for a majority of the length of one or both of the intermediate portions only extend through the first and second outer layers 20, 40 and not extend through the middle woven layer 60. This may for the entire length of one or both of the intermediate portions, or may be for an overwhelming majority (e.g. 80% or more) of the length of the intermediate portions 140, 160.

The term “line” of stitching 240 is defined herein to mean a continuous stitch. Portions of the line may be straight and portions of the line may be curved to allow the line 240 to extend about the perimeter of the device 10 as discussed herein. The line segments that are discussed above that extend through different portions of the device may also be straight and have curved portions to allow those line segments extend about the perimeter of the device.

The line 240 may start (i.e. the location where the first stitch is provided) at various different locations within the device 10, such as proximate to one of tips 180a, 200a of a respective end portion 180, 200, or within an intermediate portion 140, 160, or along the central portion 110. Because the line 240 extends along the entire perimeter (or substantially the entire perimeter—in a case where a final stitch of a line ends just short of the initial stitch of the line as the final stitch returns to proximate to the initial stitch) the initial stitch may occur at virtually any position along the device. In some embodiments, an initial stitch 241 (shown schematically FIGS. 7a-7c) may occur at an end portion 180, 200, or along the central portion 110 such that the initial stitch extends through all three layers, as discussed herein. In some embodiments, for ease of assembly and stitching the three layers 20, 80, 40 (in the order that they are arranged) may be placed within tooling (not shown, but can be understood features of appropriate tooling by one of ordinary skill after a thorough review of this specification) such that the location of the initial stitch is identified by the construction of the tooling. In some embodiments, the line 240 may extend clockwise along the perimeter of the device 10 (from the perspective of the depicting of the device in FIG. 2), while in other embodiments, the line may extend counter-clockwise along the perimeter of the device 10. One of ordinary skill in the art with a thorough review and understanding of this disclosure will appreciate that the stitching patterns may vary within the above discussed patterns, and that a suitable pattern would be chosen for the desired ease of assembly, strength, or other factors.

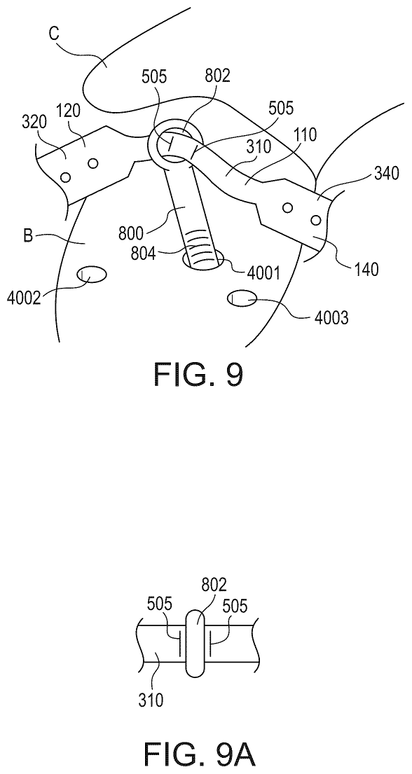

In some embodiments, like those depicted in FIG. 1a, include two stitched lines 505 that are perpendicular to the longitudinal axis 1002 of the device 10 may be provided through the central portion 310 and specifically both spaced away from the geometric center of central portion a short distance, such as 1.5 mm, so that the two lines 505 are about 3.0 mm apart (plus or minus 1.0 to 2.0 mm). These lines 505 are provided to allow for ease of placement of the device through the eyelet 802 of an anchor 800 that is intended to receive the central portion 310 of the device when implanted. When positioning the device, the physician would place the device such that both lines 505 reside outside of an on opposite sides of the eyelet 802, as depicted schematically in FIGS. 9 and 9a.

In some embodiments, additional perpendicular stitched lines Y may be provided, such as on the intermediate portions, and can be used for visual indicators for proper positioning when used clinically for various clinical procedures.

The first and second outer layers 20, 40 are preferably formed from the same construction and the same geometry. In other embodiments, the first and second outer layers 20, 40 may have the same geometry with respect to a plane that extends along the length and width directions but may be formed with different material thicknesses.

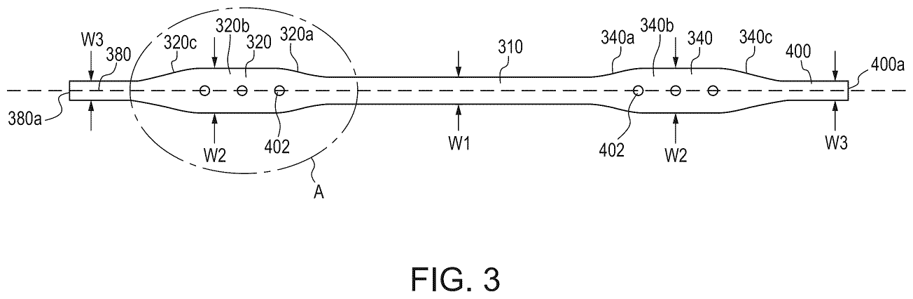

The first and second layers 20, 40 are formed as depicted in FIG. 3 (the outer layer 340 is the same as the outer layer 320 depicted in the figure). The layers include the central portion 310, first and second intermediate portions 320, 340, and first and second end portions 380, 400, with these portions of the layers 20, 40 corresponding to the portions 110, 120, 140, 180, 200 of the overall assembled device as discussed herein. The central portion 310 is arranged with a constant width W1 along its length. The first and second intermediate portions 320, 340 include respective constant width portions 320b, 340b and transition portions (320a, 340a) that transition from the central portion 310 to the constant width portion (320b, 340b) of the intermediate portion. The first and second intermediate portions 320, 340 additionally include second transition portions (320c, 340c) that transition from the constant width portion (320b, 340b) to the adjacent first and second end portions 380, 400. In some embodiments, the transition portions 320a and 320c have the exact same or substantially the same curvature (FIG. 6) as the width of the layer transitions with respect to the constant width W2 of the intermediate portion, while in other embodiments the transition portions 320a and 320c may have differing curvatures (FIG. 5). For example, as depicted in FIG. 5, the transitions 320a, 320c have the same curvature because the widths of the central portion (W1) and the end portion W3 are the same or substantially the same. In the embodiment of FIG. 5, the end portion width W3 is significantly smaller than the width W1 of the central portion and therefore the transition 320c is substantially more abrupt or sloped than the transition 320a.

The constant width portions 320b, 340b of the first and second intermediate portions have a width W2 that are larger than the width along the entire central portion and the width along the entire first and second end portions 380, 400.

In some embodiments, the width W1 of the central portion 310 is larger than the width W3 of the entire first and second end portions 380, 400. In other embodiments, the width W1 of the central portion 310 is larger than a width W3 majority of the first and second end portions 380, 400, but a portion of each of the first and second end portions is either the same as the width of the central portion 310 or larger than a width of the central portion.

The width W3 of the first and second end portions 380, 400 at the tip 380a, 400a of the respective end portion is less than the width W1 of the central portion 310.

In some embodiments, FIGS. 7a-7c, the first and second end portions 380, 400 have a constant width (after the transition portions 320c, 340c) until reaching the respective tip 380a, 400a. In some embodiments, the tip 380a, 400a is a surface that is perpendicular to the constant width sides 381b, 381c, 401b, 401c of the respective end portion. In other embodiments, the tip 380a, 400a is a curve, such as semi-circular.

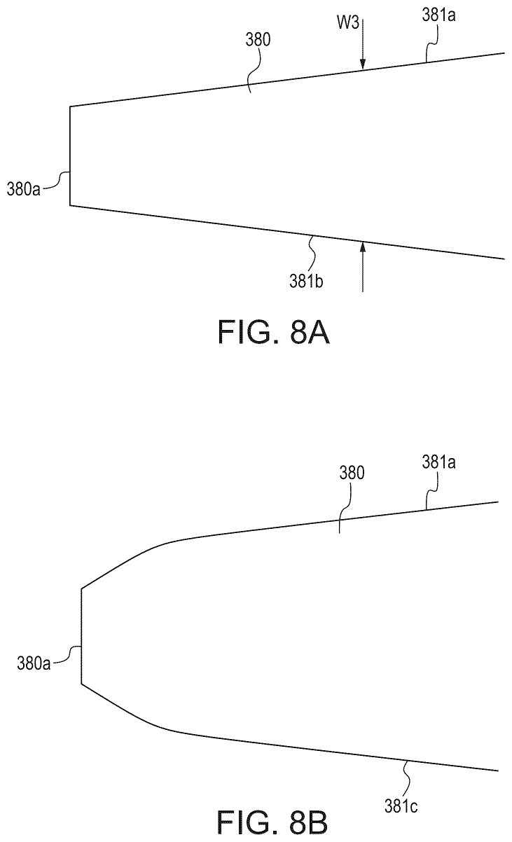

In other embodiments, FIGS. 8a-8b, the first and second end portions 380, 400 have a changing (typically decreasing) width along their length in a direction from adjacent to the neighboring intermediate portion 320, 340 toward the respective tip 380a, 400a. In this embodiment, the sides 381a, 381b, 401a, 401b may be straight before reaching the tip, while in other embodiments the sides may be curved, while in still other embodiments the sides may have straight and curved portions.

In some embodiments, the outer layers 20, 40 may have one or a plurality of thru holes 402 that are disposed within one or both of the intermediate portions 320, 340. In embodiments where three or more through holes 402 are provided, all through holes 402 may be equidistant from their adjacent through holes 402. In embodiments where through holes 402 are provided, the woven layer 80 beneath the through hole is exposed through the respective first or second layer 20, 40.

The first and second layers 20, 40 are formed at least partially from a collagen material that serves as a scaffold. In some embodiments the collagen may be partially cross-linked bovine pericardium. Various collagen materials that are appropriate for use as the first and second layers 20, 40 are disclosed in U.S. Pat. Nos. 8,901,078; 9,220,808; 9,399,084; 9,592,320; and 10,611,822 that are incorporated by reference herein in their entirety.

As utilized herein, the term ‘scaffold’ can generally refer to biocompatible materials that can facilitate cellular growth and development when located in proximity to living cells. Scaffold materials encompassed herein include those designed for in vivo, ex vivo, and/or in vitro use. In general, scaffold materials can describe a physical structure that can allow cellular ingrowth to the scaffold. For example, a scaffold can include macro- and/or microporosity that can allow cellular and/or nutrient propagation throughout all or a portion of the scaffold. In one embodiment, a scaffold can include a matrix with a mesh size, ξ, or a pore size, ρ, which can allow cellular propagation, nutrient propagation, and/or ingrowth throughout the matrix. Scaffolding materials as may be included in disclosed devices can include those disclosed in U.S. patent application Ser. No. 11/777,733, to Brunelle, et al., incorporated herein in its entirety by reference, as well as U.S. Pat. No. 9,387,280 to Brunelle, incorporated herein in its entirety by reference.

Scaffolds encompassed by the disclosed subject matter can include one or more materials that can encourage the growth and development of a cellular construct. For instance, a scaffold can include one or more synthetic or natural biocompatible polymers that have been shown to promote wound healing.

Biocompatible synthetic polymers as may be utilized in forming a scaffold can include, e.g., polyurethanes, polyesters, polyethylenes, silicones, polyglycolic acid (PGA), polylactic acid (PLA), copolymers of lactic and glycolic acids (PLGA), polyanhydrides, polyorthoesters, and the like. A scaffold can include one or more natural polymers including, e.g., chitosan, glycosaminoglycans, and collagen.

In one preferred embodiment, the scaffold can contain collagen. Collagen is the most abundant fibrous structural protein found in mammals and has been shown to exhibit many desirable qualities in scaffolding materials. For example, in addition to good bioaffinity and histocompatibility, wound healing cells such as fibroblasts have been shown to have good affinity for collagen, and the presence of collagen in a scaffold can encourage and promote cell growth and differentiation of the tissues/cells associated with the scaffold. In addition, collagen can act as a conduit for healthy cells and nutrients from surrounding healthy tissue such as healthy tendon or bleeding bone to the repair site.

Collagen encompassed by the present disclosure can include any collagen type or combination of collagen types. For instance, a collagen-containing scaffold can include any one or combination of the currently known 28 types of collagen. Typically, a collagen-containing scaffold can include at least some type I and/or type II collagen, as types I and II collagen are the most abundant types of collagen, and the introduction of organized type I collagen has been shown to be beneficial in cellular integration and tendon remodeling. However, it should be understood that the presence of either of any specific collagen type is not a requirement in a collagen-containing scaffold as disclosed herein.

A collagen-containing scaffold can be derived of any suitable collagen source and formed according to any suitable method as is understood by one of ordinary skill in the art. For example, a collagen-based scaffold can include natural collagen-containing tissues that can be allograft, autograft, and/or xenograft tissues. Natural collagen-containing tissues that can be used to form a scaffold can include, without limitation, soft tissues including ligament, tendon, muscle, dura, pericardium, fascia, peritoneum, and the like and can be derived from any host source (human, equine, porcine, bovine, etc.).

A natural tissue scaffold can be processed to remove some or all of the cellular components of the tissue. For example, a tissue for use as a scaffold can be air-dried or lyophilized to kill cells contained therein. Thermal shock, sonication or ultrasound treatment, changes in pH, osmotic shock, mechanical disruption, or addition of toxins can also induce cell death or apoptosis. Other treatments to de-cellularize or denature the tissue are possible using radiation, detergents (e.g., sodium dodecyl sulfate (SDS)), enzymes (RNAase, DNAase), or solvents (alcohol, acetone, or chloroform). These techniques are only some of the examples of techniques to de-cellularize, denature or chemically modify all or part of the tissue and are not meant to limit the scope of the disclosure. For example, methods of de-cellularizing can utilize, for example, enzymes such as lipases combined with other enzymes and, optionally, detergents. Treatment with hypotonic and/or hypertonic solutions, which have non-physiological ionic strengths, can promote the de-cellularization process. These various de-cellularization solutions generally are suitable as treatment solutions. Proteases also can be used effectively to de-cellularize tissue. The de-cellularization can be performed in stages with some or all of the stages involving differential treatments. For example, a mixture of proteases, nucleases and phospholipases can be used in high concentrations to de-cellularize a tissue.

Collagen-containing materials can be processed according to any suitable methods during a collagen scaffold preparation process. For instance, a collagen-containing scaffold can be derived from reconstituted collagen. The capability of utilizing reconstituted collagen to form a scaffolding material was first published by Bell, et al. in 1979 (Proc. Natn. Acad. Sci. USA, 76, 1274-1278, incorporated herein by reference). In general, methods for forming scaffolds from reconstituted collagen include extraction and purification of collagen(s) from connective tissues by solubilization that can be acidic, alkaline, neutral and/or enzymatic in nature. The extracted collagen can be broken down to monomeric and/or oligomeric level and stored as a powder or liquid. Upon rehydration, a solution can form that can be molded and crosslinked via chemical or physical methods to form a scaffold.

Variations and improvements upon these processes can be utilized. For example, U.S. Pat. No. 6,623,963 to Muller, et al., incorporated herein by reference, describes a method for forming a scaffold that includes solubilizing animal cartilage tissue by physical and/or chemical treatment processes that include treatment with various buffers to remove impurities and to separate the solid and liquid phases; physical treatment to separate solid and liquid phases, such as by centrifugation; and treatment with a proteolytic enzyme that breaks the crosslinking of the collagen in its telopeptide region into its virtually non-crosslinked, atelocollagen, triple helix form. The collagen thus obtained is then reconstituted, i.e., the non-crosslinked, atelocollagen form of collagen reestablishes its crosslinking between the variable regions along the collagen molecule, including some remaining residues in the telopeptide region. As a result, the solubilized collagen loses its liquid or gel-like consistency and becomes more rigid with a higher degree of structural integrity such that it may be utilized as a scaffold.

The material may include collagen fibers free of the immunogenic, telopeptide portion of native collagen. The telopeptide region provides points of crosslinking in native collagen. Specifically, collagen obtained from tendons, skin, and connective tissue of animals, such as a cow, is dispersed in an acetic acid solution, passed through a meat chopper, treated with pepsin to cleave the telopeptides and solubilize the collagen, precipitated, dialyzed, crosslinked by addition of formaldehyde, sterilized, and lyophilized. The disclosed method can obtain the atelocollagen form of collagen, free from non-collagen proteins, such as glycosaminoglycans and lipids. Further, the collagen may be used as a gel to make, for example, a membrane, film, or sponge and the degree of crosslinking of the collagen can be controlled to alter its structural properties.

Of course, the above described methods and resulting products are merely embodiments of processing as may be carried out in forming a collagen-containing scaffold as may be utilized in forming a composite device as disclosed herein and the present disclosure is in no way limited to these embodiments. Many other processing methods and scaffolds formed thereby are known to those of ordinary skill in the art and thus are not described at length herein, any of which may be utilized according to the disclosure.

Moreover, the presently disclosed subject matter is not limited to collagen scaffolds. For instance, in one embodiment, a scaffold can include or be formed entirely of a non-collagen hydrogel matrix. Hydrogel scaffolds are known in the art and are generally defined to include polymeric matrices that can be highly hydrated while maintaining structural stability. Suitable hydrogel scaffolds can include non-crosslinked and crosslinked hydrogels. In addition, crosslinked hydrogel scaffolds can optionally include hydrolyzable portions, such that the scaffold can be degradable when utilized in an aqueous environment. For example, in one embodiment, a scaffold can include a cross-linked hydrogel including a hydrolyzable cross-linking agent, such as polylactic acid, and can be degradable in an aqueous environment.

Hydrogel scaffolds can include natural polymers such as glycosaminoglycans, polysaccharides, proteins, and the like, as well as synthetic polymers, as are generally known in the art. A non-limiting list of polymeric materials that can be utilized in forming hydrogel scaffolds, in addition to collagen, previously discussed, can include dextran, hyaluronic acid, chitin, heparin, elastin, keratin, albumin, polymers and copolymers of lactic acid, glycolic acid, carboxymethyl cellulose, polyacrylates, polymethacrylates, epoxides, silicones, polyols such as polypropylene glycol, polyvinyl alcohol and polyethylene glycol and their derivatives, alginates such as sodium alginate or crosslinked alginate gum, polycaprolactone, polyanhydride, pectin, gelatin, crosslinked proteins peptides and polysaccharides, and the like.

Hydrogel scaffolds can be formed according to any method as is generally known in the art. For instance, a hydrogel can self-assemble upon mere contact of the various components or upon contact in conjunction with the presence of particular external conditions (such as temperature or pH). Alternatively, assembly can be induced according to any known method following mixing of the components. For example, step-wise or chain polymerization of multifunctional monomers or macromers can be induced via photopolymerization, temperature dependent polymerization, and/or chemically activated polymerization. Optionally, a hydrogel can be polymerized in the presence of an initiator. For example, in one embodiment, a hydrogel scaffold can be photopolymerized in the presence of a suitable initiator such as Irgacuree or Darocur® photoinitiators available from Ciba Specialty Chemicals. In another embodiment, a cationic initiator can be present. For example, a polyvalent elemental cation such as Ca²⁺, Mg²⁺, Al³⁺, La³⁺, or Mn²⁺ can be used. In another embodiment, a polycationic polypeptide such as polylysine or polyarginine can be utilized as an initiator.

A scaffold may be processed as desired prior to forming a composite device. For instance, a natural or reconstituted tissue can be stabilized through crosslinking. Generally, a stabilization process operates by blocking reactive molecules on the surface of and within the scaffold, thereby rendering it substantially non-antigenic and suitable for implantation. In 1968, Nimni et al. demonstrated that collagenous materials can be stabilized by treating them with aldehydes. (Nimni et al., J. Biol. Chem. 243:1457-1466 (1968).) Later, various aldehydes were tested and glutaraldehyde was shown to be capable of retarding degeneration of collagenous tissue. (Nimni et al., J. Biomed. Mater. Res. 21:741-771 (1987); Woodroof, E. A., J. Bioeng. 2:1 (1978).) Thus, according to one embodiment, a glutaraldehyde stabilization process as is generally known in the art may be utilized in forming a scaffold (see, e.g., U.S. Pat. No. 5,104,405 to Nimni, which is incorporated herein by reference).

A glutaraldehyde process is only one potential processing method, however, and a scaffold material processed according to any other method as is known in the art may alternatively be utilized. For example, a scaffold material as may be utilized in a disclosed composite device can be stabilized according to a physical crosslinking process including, without limitation, radiation treatment, thermal treatment, electron beam treatment, UV crosslinking, and the like.

In one preferred embodiment, a scaffold can be processed according to a non-glutaraldehyde crosslinking process. For example, non-glutaraldehyde crosslinking methods as disclosed in U.S. Pat. Nos. 5,447,536 and 5,733,339 to Girardot, et al., both of which are incorporated herein by reference, can be utilized. According to one such embodiment, a collagen-containing scaffold can be crosslinked via formation of amide linkages between and within the molecules of the scaffold. For instance, di- or tri-carboxylic acids and di- or tri-amines of about six to eight carbon atoms in length can be used in a sequential manner to form amide crosslinks.

Optionally, a scaffold can be formed to include additional materials. For instance, cellular materials can be retained in or loaded into a scaffold. For example, chondrocytes and/or fibroblasts can be retained in a natural tissue scaffold or loaded into a scaffold prior to implantation. In one embodiment, a scaffold can be seeded with cells through absorption, cellular migration, physical cyclic loading, and scaffold tensioning, optionally coupled with application of pressure through simple stirring, pulsatile perfusion methods or application of centrifugal force. In general, cell seeding can usually be carried out following combination of a scaffold with the other components of the device, described in more detail below.

Other materials as may be incorporated into disclosed composite devices via a scaffold can include any other additive as is generally known in the art. For instance, biologically active agents such as growth factors, antibiotics, extra cellular matrix components, or any other chemical or biological agent as may be beneficially incorporated into a scaffold is encompassed by the presently disclosed subject matter. Additional materials can be loaded into a scaffold, applied to a surface of a scaffold, or combined with another component of a device, as desired.

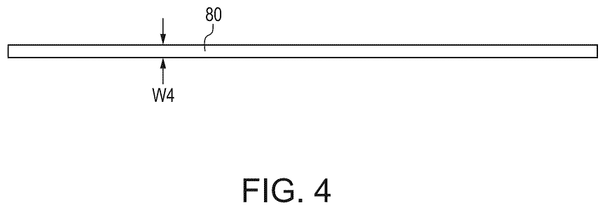

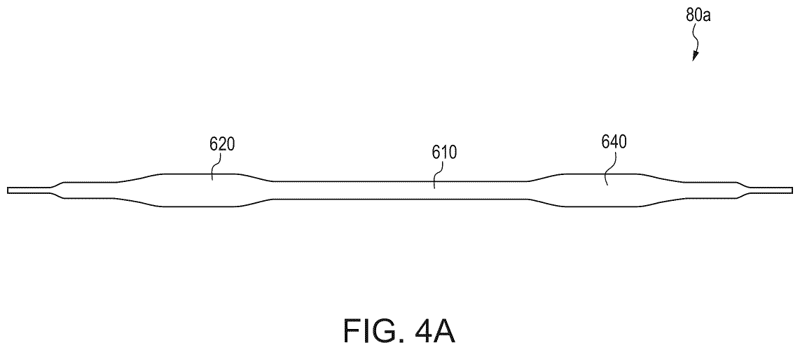

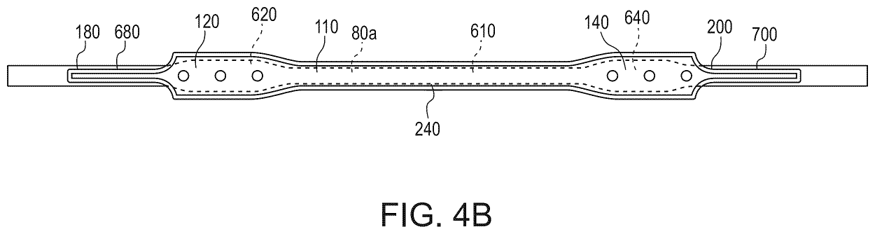

A first embodiment of a woven layer 80 is depicted in FIG. 4 and an alternate embodiment of a woven layer 80a is depicted in FIG. 4a. The woven layer 80, 80a is positioned between the first and second outer layers 20, 40. The woven layer 80, 80a may be formed of a woven construct. In some embodiments, the woven layer may be formed from woven HTPET (high tenacity polyester) fibers.

In some embodiments as depicted in FIG. 4, the woven layer 80 is formed from a constant width W4 along its length. In these embodiments, the width of the woven layer 80 has a width that is either the same or just smaller than the width W1 of the central portion 310 of the first and second layers 20, 40. In these embodiments, the width of the woven layer 80 is less than the width W2 of the intermediate portions 320, 340 of the first and second layers 20, 40. Specifically, the width W2 of the intermediate portions 320, 340 may be greater than the width W4 of the woven layer 80 through the entire intermediate portion. In this embodiment, the transitions that extend toward and away from the constant width portions 320b, 340b of the intermediate portions 320, 340 are also wider than the width W4 woven layer 80.

In some embodiments, the width W4 of the woven layer 80 is greater than the width W3 of at least a portion of each of the first and second end portions 380, 400, such that a portion of the woven layer 80 that extends in the width directions past the edges of the first and second end portions 380, 400 is exposed. The woven layer 80 may extend longitudinally past the respective tips 380a, 400a of the first and second end portions 380, 400. In this embodiment, the woven layer 80 is implemented when the device 10 is deployed to pull the device through the eyelets 802 of the bone anchors 800 that are used to fix the device 10 in place.

In other embodiments as depicted in FIG. 4a, the woven layer 80a has a varying width along it length, with a central portion 610, first and second intermediate portions 620, 640, and first and second end portions 680, 700, with these portions corresponding to the portions of the overall assembled device such that the portions of the woven layer 80 are disposed within the like named portions of the overall device—i.e. the central portion 610 of the woven layer 80a is disposed within the central portion 110 of the overall device, the first intermediate portion 620 of the woven layer 80a is disposed within the first intermediate portion 120, and so on.

The woven layer 80a, and specifically at least the central portion 610 and the first and second intermediate portions 620, 640 are formed with a smaller width than the width of the layers 20, 40 along the same portions. FIG. 4b depicts the overall device with the dimensions of the woven layer 80a in dashed lines to depict the relative sizes. In some embodiments, the woven layer 80a is sized such that the line of stitching 240 of the assembled device is between the two outer layers 20, 40 and the woven layer 80a is inboard of the line of stitching, at least within the first and second intermediate portions 120, 140 (FIG. 4b) and in some embodiments also within the central portion 110. In these embodiments, the line of stitching 240 extends through the two outer layers 20, 40 and the woven layer 80a within all or a portion of the first and second end portions 180, 200. In this embodiment, the width of the woven layer 80a within the first and second intermediate portions 620, 640 is greater than the width within the central portion 610. In this embodiment, the width of the first and second intermediate portions 620, 640 of the woven layer 80a is larger than the width of the central portion 110 of the first and second layers 20, 40.

In some embodiments, the device 10 may be formed such that it has the same geometry on both sides of a line 1001 in the width direction that crosses the central portion 310 through the center of the device 10 in the length direction. The device may additionally or alternatively be formed such that it has the same geometry on both sides of a line 1002 in the length direction through a center of the device 10.

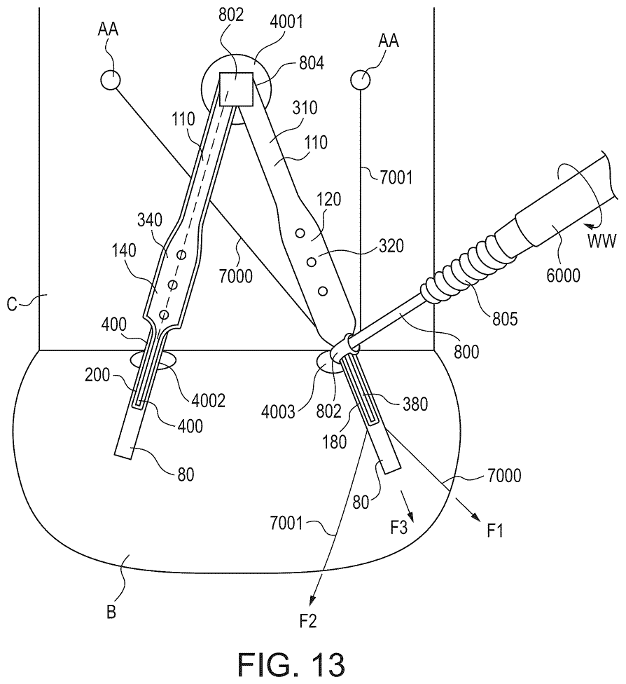

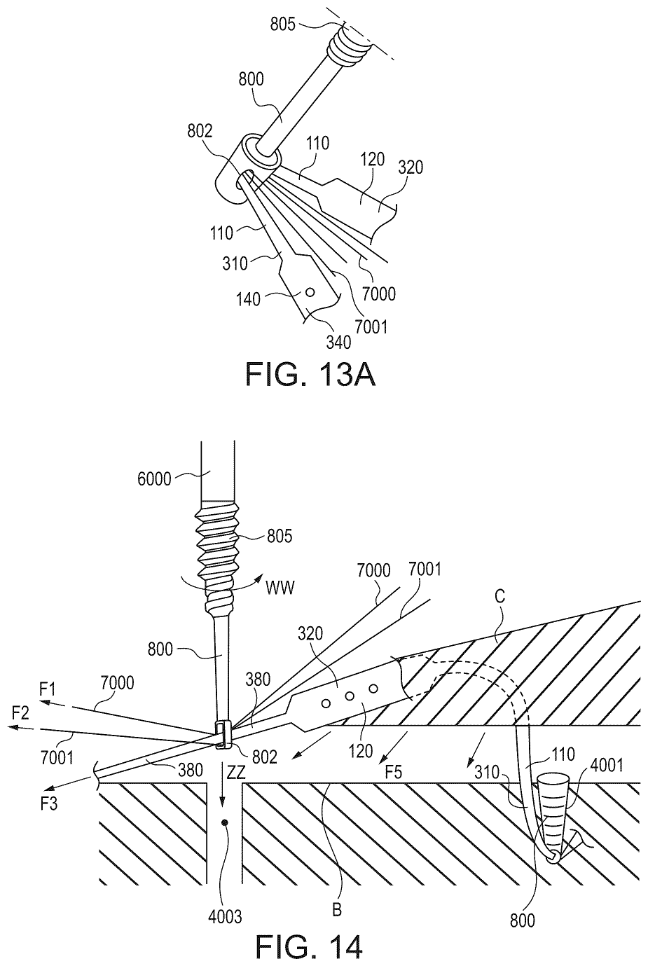

The entire device 10 may be formed such that it is sufficiently flexible that it can be pulled (in some embodiments in a rolled configuration-such that the device is rolled about its longitudinal axis, while in other embodiments in a curved orientation where the device extends along the curvature of all of or a majority of the circumference of the eyelet but does not overlap itself as in a rolled configuration) through an eyelet of a conventional bone anchor (800), such as conventional anchors that are known to be configured to be used within the scapula and humerus associated with a rotator cuff repair procedure. In other embodiments, a modified bone anchor is provided with a larger eyelet than a conventional size (discussed herein) is used to retain the center portion 110 of the device due to the relatively large diameter of the rolled or curved central portion 110 (FIG. 13a, schematic) as well as, in some embodiments, one or more sutures, or tapes (e.g. 7000, 7001). The anchors 800 that retain the end portions 180, 200 of the device (such as in lateral holes 4003, 4002—as discussed below with respect to the double row fixation technique) can have conventional sized eyelets due to the much smaller size of the end portions 180, 200 of the device in comparison to the central portion 110. Specifically, the eyelet of the anchor that receives the central portion 110 of the device must be sized to allow the rolled or curved intermediate portion 140, 160 to be pulled therethrough and retain the rolled or curved central portion 110, which can be understood to have the largest rolled or curved diameter due to the larger width of the intermediate portion 140, 160 when compared to the central portion 110, as well as when compared to the end portions 180, 200 (compare widths W2, W1, W3-FIG. 3).

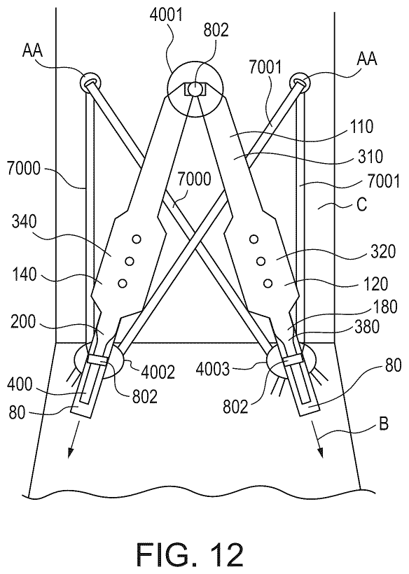

In some embodiments, the rolled or curved device can fit through an eyelet of a bone anchor (discussed herein) as well as two lines of suture also extending through the eyelet (as used when the device 10 is deployed in parallel with a double row fixation technique as depicted in FIG. 12) Potential eyelets that could be used to receive and fix the device may have cross-sectional opening areas of between 5 and 8 mm2, and may be sized to receive several sutures (such as #2 sutures therethrough, such as 4, 6, or 8 #2 sutures. As is known in the art, the cross-sectional area of one #2 suture is between 0.785 and 1.127 mm2, and the cross-sectional area of eight #2 sutures is between 6.286 and 9.018 mm2. Conventional devices, which have geometries larger than the device 10 may not be able to fit through conventional eyelets without significant stretching during threading.

In preferred embodiments, end portions 180, 200 of the first and second layers 20, 40 is formed with a width that narrows along the length of the end portion from the transition portions that continue from the adjacent intermediate portion. In some embodiments, the width of the first and second end portions (upon the first and second layers 20, 40) narrows continuously to a minimum width at the tip of the end portion. In some embodiments, the woven layer may be at a constant diameter along the first and second end portions of the device (i.e. the woven layer 80). In some embodiments, the woven layer 80, 80a may have a larger width than the first and second layers 20, 40 along all or a portion of the respective first and second end portions 180, 200 all the way to the tip of each end portion. This construction (i.e. with a narrower first and second layer 20, 40 than the woven layers, as well as in some embodiments, with a narrowing width of the first and second layers 20, 40 within at least portions of the first and second end portions) allow for the device to be at a minimum cross-sectional area at the tip of the respective end portion that is desired to be threaded through an eyelet of a suture anchor—for easier threading. The narrowing and the overall construction of the first and second layers 20, 40 and the woven layer 80, 80a within the first and second end portions also has been found to facilitate rolling the device about its longitudinal axis to facilitate threading the device 10 through eyelets during a deployment procedure.

The end portions 380, 400 and the central portion 310 may be further formed with a width (e.g. 3.0 mm maximum width) such that these features can extend through an eyelet of a conventional bone anchor when the device is in the desired position with the central portion 310 of the device 10 extending through the eyelet in an exactly flat orientation or in a substantially flat orientation. The term substantially flat is defined herein to mean exactly planar and include a range of up to 20 degrees of curvature from the planar configuration. The sizing of the width of the central portion 310 and the first and second end portions 380, 400 is adapted such that when the device is positioned (e.g. in a medial fixation technique or a double row fixation technique) that the intermediate portions 340, 360 are capable of being arranged in a planar orientation (i.e. to be fully extended and rest against the anatomical feature that the intermediate portion 320, 340 contacts, and follow any curvature of the anatomical feature) almost immediately as the material that extends through each eyelet.

In a representative embodiment, the device 10 has the following dimensions. The central portion 310 has a length of preferably 40 mm and may include any dimension within a range of 35 to 45 mm inclusive of all dimensions within this range. The central portion has a width W1 of preferably about 3 mm and may include any dimension within the range of 1 to 5 mm inclusive of all dimensions within this range. The intermediate portions 340, 360 and specifically the constant width portions of the intermediate portion preferably has a width W2 of about 7 mm and may include all widths within the range of 5 mm to 9 mm including all dimensions within this range. The Intermediate portions, and specifically the constant width portions of the intermediate portion each nominally have a length within the range of 12 to 25 mm including all lengths within this range, and such as about 15 mm, 18 mm, 21 mm, and 24 mm. In embodiments where thru holes 402 are provided, the thru holes may be spaced 6.0 mm apart from adjacent through holes, and in a preferred embodiment the thru holes have a diameter of 1.6 mm (0.3 mm tolerance). The first (i.e. furthest from the tip of the layer) thru hole 402 may be 28 mm from a geometric centerline of the layer. The overall length first and second outer layers 20, 40 may be about 125 mm. The ends may extend about 18.5 mm (tolerance 3 mm) from the end of the constant width portion 322a, 342b closest to the tip of the layer 20, 40.

The device 10 is formed with first and second layer 20, 40 and woven layer 80 construction that is sufficiently flexible to be capable of being rolled tightly or in a curved configuration to extend along the circumference of the eyelet, as needed for the intermediate portions 320, 340 to extend through an eyelet of an anchor and to avoid kinking or creasing or being deformed permanently in any fashion as the device is rolled up or curved and then released from an eyelet.

FIGS. 9-11 are schematic views of various steps of deploying the device 10 in order to fix a tissue (C) with respect to a bone. These figures when understood by one of ordinary skill in the art and with further reference to the remainder of the specification are applicable in corrective rotator cuff procedures, and will be understood to be similarly applicable for use of the device (with possible dimensional changes as needed for the desired clinical anatomy) with respect to other clinical areas where a tissue is to be fixed to a bone, with various examples of other clinical situations discussed herein.

FIG. 9 depicts a bone anchor 800 being positioned to be inserted into a hole 4000 in a bone B, with a central portion 310 of the device (110-310 of the layer 20 is depicted in the figure) threaded through an eyelet 802 of the bone anchor. The central portion 310 (110) is understood to be properly aligned because the lateral stitches 505 through the central portion 310 (110) are disposed outboard of the opposite sides of the eyelet, as confirmed by the detail top view of FIG. 9a. The first and second intermediate portions 320, 340 (120, 140) of the device extend outward and are ready to be further implemented. The bone anchor 800 may be a specialized anchor with a larger eyelet than a conventional eyelet that is configured for receipt of one or several sutures or tapes, the eyelet is sized to receive the entire rolled or curved device 10 therethrough, which has a diameter, when tightly rolled or curved, that is more than several sutures or tapes. One of ordinary skill in the art would be able to choose an anchor with an eyelet that is sufficient for the tightly rolled or curved device 10 to be pulled through with a knowledge of anchors that are clinically available and the knowledge of the diameter of the rolled or curved device.

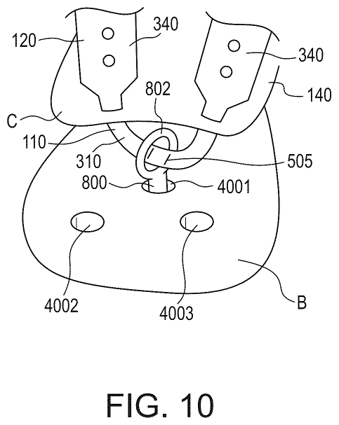

FIG. 10 depicts the bone anchor 800 being fixed within the bone B, and the two sides of the device having been pulled through different portions of the tissue C, with the tissue C pulled such that the central portion 310 (110) of the device 10 extends through both holes in the tissue and the first and second intermediate portions 320, 340 start shortly after the device extends through the tissue.

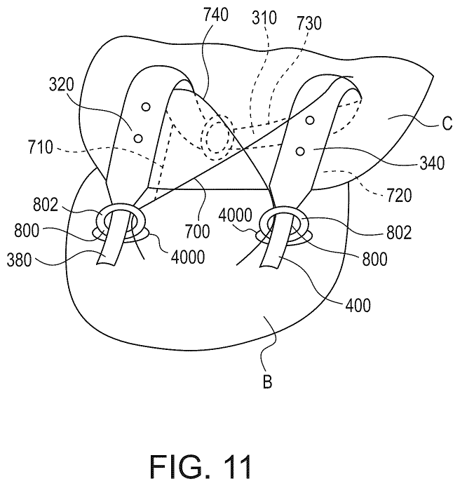

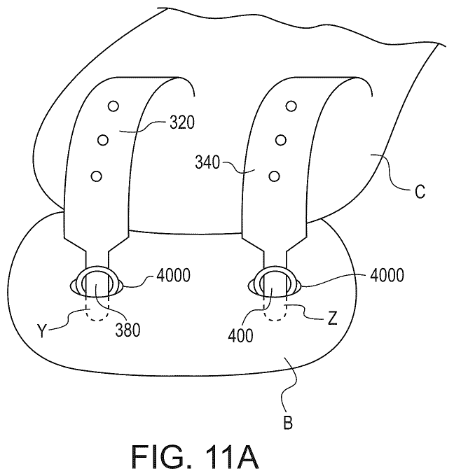

FIG. 11 depicts the device 10 being clinically fixed with respect to the bone, with a medial fixation technique. The first and second intermediate portions 320, 340 (120, 140) extend along the outer surface of the tissue and the transitions between the intermediate portions and the end portions 380, 400 (180, 200) are proximate the edge of the tissue C. Each end portion 380, 400 extends through an eyelet 802 of a bone anchor 800, which are both received within a hole 4000 in the bone B. The hole 4000 that receives the anchor 800 that receives the central portion 310 of the device is depicted in broken lines through the tissue C. The ends of the first and second end portions 380, 400 (180, 200) which may be only the woven layer 80/80a, or may be both the first and second layers 20, 40 and the woven layer 80 extend from the anchors 800. FIG. 11 depicts sutures 700, 710, 720, 730, 740 that extend through the tissue C (preferably the same tissue holes through which the device 10 extends) that form a crossing pattern and are also fixed to the anchors 800. The sutures provide a second structure, independent of the device 10, to fix the tissue C to the bone B.

FIG. 11a depicts a similar final position to FIG. 11, but the first and second end portions 380, 400 (180, 200) of the device 10 extend within the bone holes 4000 that receive the bone anchors to cause direct contact between the device 10, and specifically the first and second layers 20, 40 and the bone B, for the benefits discussed herein.

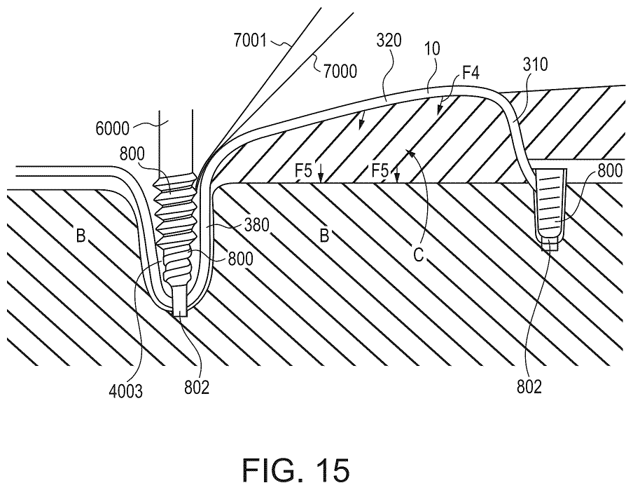

Turning now to FIGS. 12-15, a method of using the device 10 in a double row fixation technique, such as to repair a damaged rotator cuff, but also usable in other anatomical situations where it is desired to partially or fully reattach a muscle or tissue to a bone is provided. With reference to FIG. 12, the double row fixation technique fixes the device 10 to the bone at three different locations, a center aperture 4001, which supports the central portion 110 (310 of the first layer 20), and offset lateral and proximal apertures 4003, 4002, which each receive respective end portions 180, 200 (380, 400) of the device. The device 10, and specifically the central portion 310 extends through the tissue (C) to be fixed to the bone shortly after it leaves the center aperture such that the device, and specifically the intermediate portions 120, 140 rests over top of the tissue (C) and when in its final position, discussed below, imparts a force (F4, FIGS. 14, 15, schematic) upon the tissue to compress the tissue against the bone (B) to assist with the tissue naturally reconnecting with a connection with the bone (B), to allow for reapproximating (re-attaching) the tissue to the bone.

The double row fixation technique also uses two distal holes AA, that each retain sutures (7000, 7001) that are used in the double row fixation technique. Specifically, the sutures 7000, 7001 that extend from the respective medial holes AA, and are fixed to the bone B at the offset holes AA with an anchor, each includes two tails, with one tail of each suture extending through the tissue (C) and extending to the hole 4002, and the other end of suture extending through the tissue (C) and extending to the other hole 4003. The tails of the sutures 7000, 7001 are each pulled through an eyelet 802 of an anchor 800 that also receives an end portion 180, 200 of the device, such that the tails of the sutures 7000, 7001 are fixed to the bone within the offset lateral apertures. As shown in FIG. 12, one tail of each suture 7000, 7001 crosses the other as that tail extends to the offset lateral aperture 4002, 4003 that is on the opposite side of the repair as the medial hole AA that retained the suture. The other tail extends distally toward the hole 4002, 4003 that is on the same side of the repair as the offset hole AA that fixes the respective suture. The sutures are tightened to also impart a force upon the tissue C to urge the tissue onto the bone B to enhance regrowth and a new fixation of the tissue C onto the bone B.

FIG. 13 is a view of the end 180 of the device, along with the tails of both sutures 7000, 7001 threaded through an eyelet 802 in an anchor 800 and aligned to be fixed into bone hole 4003. An operator 6000 is positioned to apply torque to cause the operator to rotate (WW) to the anchor 800 to cause the anchor to screw into the lateral bone hole 4003, with the threaded portion 805. The anchor is designed with the eyelet 802 at the bottom of the anchor, such the eyelet 802 is the deepest within the hole 4003 when the anchor is fully deployed within the bone. The figure shows a force (F3) schematically applied to the end portion 180 of the device that extends through and out of the eyelet 802, as well as a force F2 applied to the suture 7001 and a force F1 applied to the suture 7000. These forces allow for the device, specifically the wider intermediate portion 120 (320) of the device and the tail of the sutures 7001, and 7000 to apply a downward force upon the tissue (C) that is retained when the anchor 800 is fixed in position, by threading the anchor downwardly into the hole 4003.

FIG. 13a is a detail view of the anchor 800 showing the central portion 110 of the device 10 threaded through the eyelet 802, as well as the two sutures 7000, 7001. Note that in the double row fixation technique of FIG. 12, sutures are typically not used through the eyelet of the anchor 800 that receives the central portion 110 of the device, although it is possible to use the device and anchors with larger eyelets 802 to receive both the rolled (or curved) device (and big enough for the rolled (or curved) intermediate portion 120, 140 to slide therethrough with one, two, or more sutures (or tapes) also extending through the eyelet.

FIG. 14 is a schematic view of the device 10 extending through the tissue (C), from where the central portion 110 is retained within the center hole 4000 by an anchor 800, with the intermediate portion 120 of one side of the device running over the tissue C, with surface to surface contact between the intermediate portion 120 and the outer surface of the tissue, with the end portion 180 being threaded through the eyelet 802 of an anchor 800 to fix to the end portion within the offset later aperture 4003 in the bone B. Two sutures 7001, 7000 are also threaded through the eyelet, and the figure depicts the tension forces upon the end 180 of the device (F3) as well as upon the two sutures (F2, F1) schematically. The figure shows that the tissue is pulled downward toward the bone B due to the tension placed upon the device as well as the sutures.

FIG. 15 is a side view depicting the device 10 at rest and applying a force (F4) upon the tissue (C), which causes the tissue to be pressed upon the bone (F5) with the anchor 800 fully seated within the hole 4003. In this final configuration, the device 10, and specifically the outer layers 20, 40 of the device make contact with the bone within the hole, which enhances tissue remodeling or regrowth via the collagen layers 20, 40 of the device. Shortly after the device is implanted as depicted in FIG. 15, the end portion 180 (also 200 not shown) of the device that extends out of the lateral hole 4003 may be cut off, as well as cutting off the ends of the sutures 7000, 7001. FIG. 15 depicts the sutures not pressed against the tissue, but in the final configuration the sutures 7000, 7001 would be tensioned so that they contact the tissue C to press the tissue into the bone, with the force enhancing tissue reconnection with the bone. In some embodiments, the sutures 7000, 7001 are above the device 10, while in other embodiments (FIG. 12) the sutures are between the device and the tissue C.

The term “about” is specifically defined herein to include a range that includes the reference value and plus or minus 5% of the reference value and all values within this range.

While the preferred embodiments of the disclosed have been described, it should be understood that the invention is not so limited and modifications may be made without departing from the disclosure. The scope of the disclosure is defined by the appended claims, and all devices that come within the meaning of the claims, either literally or by equivalence, are intended to be embraced therein.

The subject disclosure can be understood with reference to the following numbered paragraphs:

Numbered Paragraph 1: An implantable tissue attachment device, comprising
an elongate flexible member, wherein the flexible member comprises a plurality of layers that extend along a length of the device,
wherein the flexible member includes a central portion, first and second intermediate portions extending from respective opposite first and second ends of the central portion, and a first end portion that extends from the first intermediate portion and a second end portion that extends from the second intermediate portion;
wherein the elongate flexible member comprises two elongate layers that are formed at least in part from a collagen material, wherein the two elongate layers are disposed to establish opposite outer layers of the member;
wherein a woven layer is disposed between the two elongate collagen layers;
wherein the first and second intermediate portions establish a first width along a majority of their length, and wherein the first width is greater than a width of the central portion along at least a majority of its length,
wherein the first width is wider than a width of the entire woven layer.

Numbered Paragraph 2: The implantable attachment device of Numbered Paragraph 1, wherein the width of the entire woven layer is smaller than the width of the central portion.

Numbered Paragraph 3. The implantable attachment device of Numbered Paragraph 1, wherein the woven layer has a greater width within the first and second intermediate portions than within the central portion.

Numbered Paragraph 4. The implantable attachment device of Numbered Paragraph 3, wherein the width of the woven layer within the first and second intermediate portions is less than a width of the two elongate layers within the first and second intermediate portions.

Numbered Paragraph 5: The implantable attachment device of Numbered Paragraph 2, wherein the woven layer has an approximately uniform width along its entire length.

Numbered Paragraph 6: The implantable attachment device of Numbered Paragraph 3, wherein the first end portion transitions from the first intermediate portion and the width of the first end portion decreases along a portion the first end portion until reaching a minimum width, and
wherein the second end portion transition from the second intermediate portion and the width of the second end portion narrows along a portion of the second end portion until reaching a minimum width.

Numbered Paragraph 7: The implantable attachment device of Numbered Paragraph 6, wherein the minimum width of the first end portion is less than the width of the central portion along the at least the majority of the length of the central portion, and the minimum width of the second end portion of the second end portion is less than the width of the central portion along the at least the majority of the length of the central portion.

Numbered Paragraph 8: The implantable attachment device of Numbered Paragraph 7, wherein the two elongate layers do not cover a portion of the woven layer within at least part of each of the first and second end portions.

Numbered Paragraph 9: The implantable attachment device of Numbered Paragraph 1, wherein the two elongate layers and the woven layer are stitched together, wherein the line of stitching extends from the first end portion to the first intermediate portion, to the central portion, to the second intermediate portion, to the second end portion and returns to the first end portion.

Numbered Paragraph 10: The implantable attachment device of Numbered Paragraph 9, wherein the line of stitching when extending through one or both of the first and second intermediate layers does not contact the woven layer as it extends through at least a majority of the length of each of the first and second intermediate layers.

Numbered Paragraph 11: The implantable attachment device of Numbered Paragraph 1, wherein transitions between the central portion and the respective first and second intermediate portions, and transitions between the first and second intermediate portions and the respective first and second end portions are each arcuate and concave.

Numbered Paragraph 12. The implantable attachment device of Numbered Paragraph 1, wherein the first and second intermediate portions of both of the two elongate layers comprise a plurality of thru holes, such that a portion of the woven layer is exposed through each thru hole.

Numbered Paragraph 13. The implantable attachment device of Numbered Paragraph 5, wherein the woven layer is about 5.8 mm wide in portions within the first and second intermediate portions, and about 3 mm wide in the portion through the central portion, and 3 mm wide along its length, and the two elongate layers are about 7 mm wide within the first and second intermediate portions.

Numbered Paragraph 14. The implantable attachment device of any of the preceding Numbered Paragraphs, wherein the flexible member is configured to be implemented in a lateral double row fixation technique for interacting with human or animal bones.

Numbered Paragraph 15. The implantable attachment device of any of the preceding Numbered Paragraphs, wherein the flexible member is configured to be implemented with a medial fixation technique for interacting with human or animal bones, with sutures simultaneously used in technique a separate fixation technique.

Numbered Paragraph 16. The implantable attachment device of Numbered Paragraph 15, wherein the flexible member is configured to be used with multiple knotless suture anchors implanted with bones in the medial fixation technique.

Numbered Paragraph 17. The implantable attachment device of Numbered Paragraph 1, wherein the collagen material is Partially cross-linked bovine pericardium.

Numbered Paragraph 18. An implantable tissue attachment device, comprising
an elongate flexible member, wherein the flexible member comprises a plurality of layers that extend along a length of the device,
wherein the flexible member includes a central portion, first and second intermediate portions extending from respective opposite first and second ends of the central portion, and a first end portion that extends from the first intermediate portion and a second end portion that is extends from the second intermediate portion;
wherein the elongate flexible member comprises two elongate layers that are formed at least in part from a collagen material, wherein the two elongate layers are disposed to establish opposite outer layers of the flexible member;
wherein a woven layer is disposed between the two elongate collagen layers;
wherein the first and second intermediate portions establish a first width along a majority of their length, and wherein the first width is greater than a width of the central portion along at least a majority of the length of the first and second intermediate portions,
wherein the first end portion transitions from the first intermediate portion and the width of the first end portion narrows along a portion the first end portion until reaching a minimum width at an outer tip of the first end portion, and
wherein the second end portion transition from the second intermediate portion and the width of the second end portion narrows along a portion of the second end portion until reaching a minimum width at an outer tip of the second end portion.

Numbered Paragraph 19. The implantable tissue attachment device of Numbered Paragraph 18, wherein the minimum width of the first end portion is at an end tip of the first end portion, and the minimum width of the second end portion is at an end tip of the second end portion.

Numbered Paragraph 20. The implantable tissue attachment device of Numbered Paragraph 18, wherein the minimum width of the first end portion is less than the width of the central portion along the at least the majority of the length of the central portion, and the minimum width of the second end portion of the second end portion is less than the width of the central portion along the at least the majority of the length of the central portion.

Numbered Paragraph 21. The implantable tissue attachment device of Numbered Paragraph 18, wherein the elongate flexible member is constructed such that a portion that is to a left side of a plane that extends through a middle of the central portion is constructed approximately the same as a portion that is to a right side of the plane.

Numbered Paragraph 22. The implantable tissue attachment device of Numbered Paragraph 18, wherein the first width is wider than a width of the woven layer.

Numbered Paragraph 23. The implantable tissue attachment device of Numbered Paragraph 22, wherein the woven layer has an approximately uniform width along its length.

Numbered Paragraph 24. The implantable tissue attachment device of Numbered Paragraph 22, wherein the woven layer has an approximately uniform width with along a portion of its length that interacts with the first and second elongate layers.

Numbered Paragraph 25. The implantable tissue attachment device of Numbered Paragraph 24, wherein the woven layer has a different width for one or more portions of the woven layer that do not interact with the first and second elongate layers.

Numbered Paragraph 26. The implantable tissue attachment device of Numbered Paragraph 18, wherein the two elongate layers and the woven layer are stitched together, wherein the line of stitching extends from the first end portion to the first intermediate portion, to the central portion, to the second intermediate portion, to the second end portion and returns to the first end portion.

Numbered Paragraph 27. The implantable tissue attachment device of Numbered Paragraph 26, wherein the line of stitching when extending through one or both of the first and second intermediate layers does not contact the woven layer as it extends through at least a majority of the length of each of the first and second intermediate layers.

Numbered Paragraph 28. The implantable tissue attachment device of Numbered Paragraph 26, wherein the line of stitching extends within both of the first and second end portions such that the line includes first and second sections that are spaced from each other, with the first and second sections extending to a transition between the first and second sections, wherein the transition is proximate to a tip of the respective first and second end portions.

Numbered Paragraph 29. The implantable tissue attachment device of Numbered Paragraph 28, wherein the transition of the line of stitching is a point.

Numbered Paragraph 30. The implantable tissue attachment device of Numbered Paragraph 28, wherein the transition is a portion that is oblique or perpendicular to an orientation of the first and second lines extending to the end portion.

Numbered Paragraph 31. The implantable tissue attachment device of Numbered Paragraph 30, wherein the first and second sections are parallel for at least a portion of their length.

Numbered Paragraph 32. The implantable tissue attachment device of Numbered Paragraph 30, wherein the first and second sections are disposed at an acute angle with respect to each other for at least a portion of their length along each of the first and second end portions of the flexible member.

Numbered Paragraph 33. The implantable attachment device of any of Numbered Paragraphs 18-32, wherein the flexible member is configured to be implemented in a lateral row fixation technique for interacting with human or animal bones.

Numbered Paragraph 34. The implantable attachment device of any of Numbered Paragraphs 18-33, wherein the flexible member is configured to be implemented with a medial fixation technique for interacting with human or animal bones, with sutures simultaneously used in a conventional lateral row fixation technique.

Numbered Paragraph 35. The implantable attachment device of Numbered Paragraph 34, wherein the flexible member is configured to be used with multiple knotless suture anchors implanted with bones in the lateral row fixation technique.

Numbered Paragraph 36. The implantable attachment device of Numbered Paragraph 18, wherein the collagen material is Partially cross-linked bovine pericardium.

Numbered Paragraph 37: A method of repairing a tissue to bone connecting comprising:
providing an implantable tissue attachment device of any one of Numbered Paragraphs 1-36,
preparing a center bone aperture and first and second bone apertures that are each offset laterally and proximal to the central bone aperture on opposite sides of the center bone aperture;
threading a first end portion of the flexible member through an eyelet of a first anchor until central portion of the flexible member is centered through the eyelet; inserting and fixing the first anchor in the center bone aperture;
   pulling the first end portion through a first position of the tissue and pulling the second end portion through a second position of the tissue at a laterally offset position from the first position;
pulling the first and second end portions toward the respective first and second offset lateral bone apertures,
arranging the flexible member such that the respective first and second intermediate portions lie substantially flat upon spaced apart respective outwardly facing surfaces of the tissue with surface to surface contact therebetween;
threading the first end portion through an eyelet of a second anchor and inserting the second bone anchor into the first offset lateral bone aperture;
threading the second end portion through an eyelet of a third anchor and inserting the third bone anchor into the second offset later bone aperture;
simultaneously with threading the first end portion through the eyelet of the second anchor, pulling the first end portion that extends through the eyelet of the second anchor to create tension in the first intermediate portion;
simultaneously with threading the second end portion through the eyelet of the third anchor, pulling the second end portion that extends through the eyelet of the third anchor to create tension in the second intermediate portion;
fixing the second anchor into the first offset lateral bone aperture and arranging the first end portion such that at least one of the outer surfaces of the first end portion makes contact with bone within the first offset lateral bone aperture; and fixing the third anchor into the second offset lateral bone aperture and arranging the second end portion such that at least one of the outer surfaces of the second end portion makes contact with bone within the second offset lateral bone aperture.

Numbered Paragraph 38. The method of Numbered Paragraph 37, further comprising:
preparing fourth and fifth apertures distally of the center bone aperture and laterally on opposite sides of the center bone anchor,
threading a first suture line through a fourth bone anchor and fixing the fourth bone anchor to the fourth aperture such that the first suture line forms first and second tails that each extend from the fourth aperture;
threading a second suture line through the fifth bone anchor and vising the fifth bone anchor to the fifth aperture such that the second suture line forms first and second tails that each extend from the fifth aperture;
pulling the first and second tails of the first suture through a third portion of the tissue laterally of the center and pulling the first and second tails of the second suture through a fourth portion of the tissue laterally of the center on an opposite side of the center from the third portion;
threading the first tail of the first suture and the second tail of the second suture through the eyelet of the second anchor before fixing the second anchor into the second offset lateral bone aperture; and
threading the second tail of the first suture and the first tail of the second suture through the eyelet of the third anchor before fixing the third anchor into the third offset lateral bone aperture;
applying tension to the first tail of the first suture and the second tail of the second suture simultaneously with pulling the first end portion of the flexible member that extends through the eyelet of the second anchor;
applying tension to the second tail of the first suture and the first tail of the second suture simultaneously with pulling the second end portion of the flexible member that extends through the eyelet of the third anchor.

Numbered Paragraph 39. The method of any one of Numbered Paragraphs 37 or 38, further comprising cutting free ends of the first and second end portions that extend from the respective second and third apertures close to the respective second and third anchors.

Numbered Paragraph 40. The method of Numbered Paragraph 38 or Numbered Paragraph 39 when depending from Numbered Paragraph 37, further comprising cutting free ends of the first and second tails of the first and second sutures that extend out of the respective second and third apertures close to the respective second and third anchors.

Numbered Paragraph 41. The method of any one of Numbered Paragraph 38-40, further comprising positioning the first and second tails of each of the first and second sutures that pass by an intermediate portion above an outer surface of the first and second intermediate portions that faces away from the tissue.

Numbered Paragraph 42. The method of any one of Numbered Paragraphs 38-40, further comprising positioning the first and second tails of each of the first and second sutures so that the tails that pass by an intermediate portion between the intermediate portion and the tissue.

1. An implantable tissue attachment device, comprising
an elongate flexible member, wherein the flexible member comprises a plurality of layers that extend along a length of the device,
wherein the flexible member includes a central portion, first and second intermediate portions extending from respective opposite first and second ends of the central portion, and a first end portion that extends from the first intermediate portion and a second end portion that is extends from the second intermediate portion;
wherein the elongate flexible member comprises two elongate layers that are formed at least in part from a collagen material, wherein the two elongate layers are disposed to establish opposite outer layers of the flexible member;
wherein a woven layer is disposed between the two elongate collagen layers;
wherein the first and second intermediate portions establish a first width along a majority of their length, and wherein the first width is greater than a width of the central portion along at least a majority of the length of the first and second intermediate portions,
wherein the first end portion transitions from the first intermediate portion and the width of the first end portion narrows along a portion the first end portion until reaching a minimum width at an outer tip of the first end portion, and
wherein the second end portion transition from the second intermediate portion and the width of the second end portion narrows along a portion of the second end portion until reaching a minimum width at an outer tip of the second end portion.

2. The implantable tissue attachment device of claim 1, wherein the minimum width of the first end portion is at an end tip of the first end portion, and the minimum width of the second end portion is at an end tip of the second end portion.

3. The implantable tissue attachment device of claim 1, wherein the minimum width of the first end portion is less than the width of the central portion along the at least the majority of the length of the central portion, and the minimum width of the second end portion of the second end portion is less than the width of the central portion along the at least the majority of the length of the central portion.

4. The implantable tissue attachment device of claim 1, wherein the elongate flexible member is constructed such that a portion that is to a left side of a plane that extends through a middle of the central portion is constructed approximately the same as a portion that is to a right side of the plane.

5. The implantable tissue attachment device of claim 2, wherein the first width is wider than a width of the woven layer.

6. The implantable tissue attachment device of claim 5, wherein the woven layer has an approximately uniform width along its length.

7. The implantable tissue attachment device of claim 5, wherein the woven layer has an approximately uniform width with along a portion of its length that interacts with the first and second elongate layers.

8. The implantable tissue attachment device of claim 7, wherein the woven layer has a different width for one or more portions of the woven layer that do not interact with the first and second elongate layers.

9. The implantable tissue attachment device of claim 1, wherein the two elongate layers and the woven layer are stitched together, wherein the line of stitching extends from the first end portion to the first intermediate portion, to the central portion, to the second intermediate portion, to the second end portion and returns to the first end portion.

10. The implantable tissue attachment device of claim 9, wherein the line of stitching when extending through one or both of the first and second intermediate layers does not contact the woven layer as it extends through at least a majority of the length of each of the first and second intermediate layers.

11. The implantable tissue attachment device of claim 9, wherein the line of stitching extends within both of the first and second end portions such that the line includes first and second sections that are spaced from each other, with the first and second sections extending to a transition between the first and second sections, wherein the transition is proximate to a tip of the respective first and second end portions.

12. The implantable tissue attachment device of claim 11, wherein the transition of the line of stitching is a point.

13. The implantable tissue attachment device of claim 11, wherein the transition is a portion that is oblique or perpendicular to an orientation of the first and second lines extending to the end portion.

14. The implantable tissue attachment device of claim 13, wherein the first and second sections are parallel for at least a portion of their length.

15. The implantable tissue attachment device of claim 13, wherein the first and second sections are disposed at an acute angle with respect to each other for at least a portion of their length along each of the first and second end portions of the flexible member.

16. The implantable attachment device of claim 1, wherein the flexible member is configured to be implemented in a lateral row fixation technique for interacting with human or animal bones.

17. The implantable attachment device of claim 1, wherein the flexible member is configured to be implemented with a medial fixation technique for interacting with human or animal bones, with sutures simultaneously used in a conventional lateral row fixation technique.

18. The implantable attachment device of claim 16, wherein the flexible member is configured to be used with multiple knotless suture anchors implanted with bones in the lateral row fixation technique.

19. The implantable attachment device of claim 1, wherein the collagen material is Partially cross-linked bovine pericardium.

20. A method of repairing a tissue to bone connecting comprising:
providing an implantable tissue attachment device of claim 1,
preparing a center bone aperture and first and second bone apertures that are each offset laterally and proximal to the central bone aperture on opposite sides of the center bone aperture;
threading a first end portion of the flexible member through an eyelet of a first anchor until central portion of the flexible member is centered through the eyelet;
inserting and fixing the first anchor in the center bone aperture;
pulling the first end portion through a first position of the tissue and pulling the second end portion through a second position of the tissue at a laterally offset position from the first position;
pulling the first and second end portions toward the respective first and second offset lateral bone apertures,
arranging the flexible member such that the respective first and second intermediate portions lie substantially flat upon spaced apart respective outwardly facing surfaces of the tissue with surface to surface contact therebetween;
threading the first end portion through an eyelet of a second anchor and inserting the second bone anchor into the first offset lateral bone aperture;
threading the second end portion through an eyelet of a third anchor and inserting the third bone anchor into the second offset later bone aperture;
simultaneously with threading the first end portion through the eyelet of the second anchor, pulling the first end portion that extends through the eyelet of the second anchor to create tension in the first intermediate portion;
simultaneously with threading the second end portion through the eyelet of the third anchor, pulling the second end portion that extends through the eyelet of the third anchor to create tension in the second intermediate portion;
fixing the second anchor into the first offset lateral bone aperture and arranging the first end portion such that at least one of the outer surfaces of the first end portion makes contact with bone within the first offset lateral bone aperture; and
fixing the third anchor into the second offset lateral bone aperture and arranging the second end portion such that at least one of the outer surfaces of the second end portion makes contact with bone within the second offset lateral bone aperture.

21. The method of claim 20, further comprising:
preparing fourth and fifth apertures distally of the center bone aperture and laterally on opposite sides of the center bone anchor,
threading a first suture line through a fourth bone anchor and fixing the fourth bone anchor to the fourth aperture such that the first suture line forms first and second tails that each extend from the fourth aperture;
threading a second suture line through the fifth bone anchor and vising the fifth bone anchor to the fifth aperture such that the second suture line forms first and second tails that each extend from the fifth aperture;
pulling the first and second tails of the first suture through a third portion of the tissue laterally of the center and pulling the first and second tails of the second suture through a fourth portion of the tissue laterally of the center on an opposite side of the center from the third portion;
threading the first tail of the first suture and the second tail of the second suture through the eyelet of the second anchor before fixing the second anchor into the second offset lateral bone aperture; and
threading the second tail of the first suture and the first tail of the second suture through the eyelet of the third anchor before fixing the third anchor into the third offset lateral bone aperture;
applying tension to the first tail of the first suture and the second tail of the second suture simultaneously with pulling the first end portion of the flexible member that extends through the eyelet of the second anchor;
applying tension to the second tail of the first suture and the first tail of the second suture simultaneously with pulling the second end portion of the flexible member that extends through the eyelet of the third anchor.

22. The method of claim 20, further comprising cutting free ends of the first and second end portions that extend from the respective second and third apertures close to the respective second and third anchors.

23. The method of claim 21 when depending from claim 20, further comprising cutting free ends of the first and second tails of the first and second sutures that extend out of the respective second and third apertures close to the respective second and third anchors.

24. The method of claim 21, further comprising positioning the first and second tails of each of the first and second sutures that pass by an intermediate portion above an outer surface of the first and second intermediate portions that faces away from the tissue.

25. The method of claim 21, further comprising positioning the first and second tails of each of the first and second sutures so that the tails that pass by an intermediate portion between the intermediate portion and the tissue.
